(19) United States
(12) Patent Application Publication
(10) Pub. No.: US 2025/0376378 A1
(43) Pub. Date: Dec. 11, 2025

(54) CARBON NANOTUBE PRODUCTION METHOD AND CARBON NANOTUBE PRODUCTION SYSTEM

(71) Applicant: Mitsubishi Electric Corporation, Tokyo (JP)

(72) Inventors: Yoji ONAKA, Tokyo (JP); Makoto TANISHIMA, Tokyo (JP); Toshio SHINOKI, Tokyo (JP); Makoto KAWAMOTO, Tokyo (JP); Seiji NAKASHIMA, Tokyo (JP); Tomoya FUKUI, Tokyo (JP); Takumi INOUE, Tokyo (JP)

(73) Assignee: Mitsubishi Electric Corporation, Tokyo (JP)

(21) Appl. No.: 18/878,109

(22) PCT Filed: Jul. 15, 2022
(86) PCT No.: PCT/JP2022/027804

§ 371 Date: Dec. 23, 2024

(51) Int. Cl. C01B 32/162; C07C 1/12; C23C 16/26; C23C 16/448; C25B 1/23; C25B 15/08

(52) U.S. Cl. CPC C01B 32/162; C07C 1/12; C23C 16/26; C23C 16/448; C25B 1/23; C25B 15/081

(57) ABSTRACT

A carbon nanotube production method according to the present disclosure includes a carbon dioxide recovery process of recovering carbon dioxide from air by using energy, a reaction process of generating a hydrocarbon by using the recovered carbon dioxide, and a carbon nanotube synthesis process of generating a carbon nanotube by using the hydrocarbon as a raw material.

### TECHNICAL FIELD

The present disclosure relates to a carbon nanotube production method and a carbon nanotube production system.

### BACKGROUND ART

Patent Document 1 discloses a CO₂ negative emission plant that captures carbon dioxide from an exhaust of a plant in operation and discharges a gas that substantially does not contain the carbon dioxide.

### CITATION LIST

### Patent Documents

Patent Document 1: PCT International Publication No. WO2012/230045

### SUMMARY OF INVENTION

### Problem to be Solved by the Invention

In a case in which the captured carbon dioxide is stored underground or the like, long-term management is required, and thus large amounts of effort and energy are required. Patent Document 1 discloses that captured carbon dioxide is used in a botanical plant. However, in Patent Document 1, the intended use of the captured carbon dioxide is limited.

The present disclosure has been made in view of the above-described problems, and provides a carbon nanotube production method and a carbon nanotube production system, with which the intended use of recovered carbon dioxide can be expanded and carbon dioxide released into the air can be reduced.

### Means to Solve the Problem

An aspect of the present disclosure relates to a carbon nanotube production method including a recovery step of recovering carbon dioxide from air by using energy, a hydrocarbon generation step of generating a hydrocarbon by using the recovered carbon dioxide, and a carbon nanotube generation step of generating a carbon nanotube by using the hydrocarbon as a raw material.

Another aspect of the present disclosure relates to a carbon nanotube production system including a carbon dioxide recovery system configured to recover carbon dioxide from air by using energy, a hydrocarbon generation system configured to generate a hydrocarbon by using the carbon dioxide recovered by the carbon dioxide recovery system, and a carbon nanotube generation system configured to generate a carbon nanotube by using the hydrocarbon as a raw material.

### Effects of the Invention

According to the present disclosure, it is possible to provide the carbon nanotube production method and the carbon nanotube production system, with which the intended use of the recovered carbon dioxide can be expanded and the carbon dioxide released into the air can be reduced.

### BRIEF DESCRIPTION OF DRAWINGS

FIG. 1 A conceptual diagram of a carbon nanotube production method according to Embodiment 1.

FIG. 2 A block diagram of a carbon nanotube production system that produces a carbon nanotube by using the carbon nanotube production method according to Embodiment 1.

FIG. 3 A conceptual diagram of a carbon nanotube production method according to Embodiment 2.

FIG. 4 A block diagram of a carbon nanotube production system that produces a carbon nanotube by using the carbon nanotube production method according to Embodiment 2.

FIG. 5 A conceptual diagram of a carbon nanotube production method according to Embodiment 3.

FIG. 6 A block diagram of a carbon nanotube production system that produces a carbon nanotube by using the carbon nanotube production method according to Embodiment 3.

FIG. 7 A conceptual diagram of a carbon nanotube production method according to Embodiment 4.

FIG. 8 A block diagram of a carbon nanotube production system that produces a carbon nanotube by using the carbon nanotube production method according to Embodiment 4.

FIG. 9 A conceptual diagram of a carbon nanotube production method according to Embodiment 5.

FIG. 10 A block diagram of a carbon nanotube production system that produces a carbon nanotube by using the carbon nanotube production method according to Embodiment 5.

FIG. 11 A conceptual diagram of a carbon nanotube production method according to Embodiment 6.

FIG. 12 A block diagram of a carbon nanotube production system that produces a carbon nanotube by using the carbon nanotube production method according to Embodiment 6.

FIG. 13 A conceptual diagram of a carbon nanotube production method according to Embodiment 7.

FIG. 14 A block diagram of a carbon nanotube production system that produces a carbon nanotube by using the carbon nanotube production method according to Embodiment 7.

FIG. 15 A conceptual diagram of a carbon nanotube production method according to Embodiment 8.

FIG. 16 A block diagram of a carbon nanotube production system that produces a carbon nanotube by using the carbon nanotube production method according to Embodiment 8.

### DESCRIPTION OF EMBODIMENTS

Hereinafter, embodiments of the present disclosure will be described with reference to the drawings. It should be noted that the scope of the present disclosure is not limited to the following embodiments, and can be changed in any way within the scope of technological ideas of the present disclosure.

### Embodiment 1

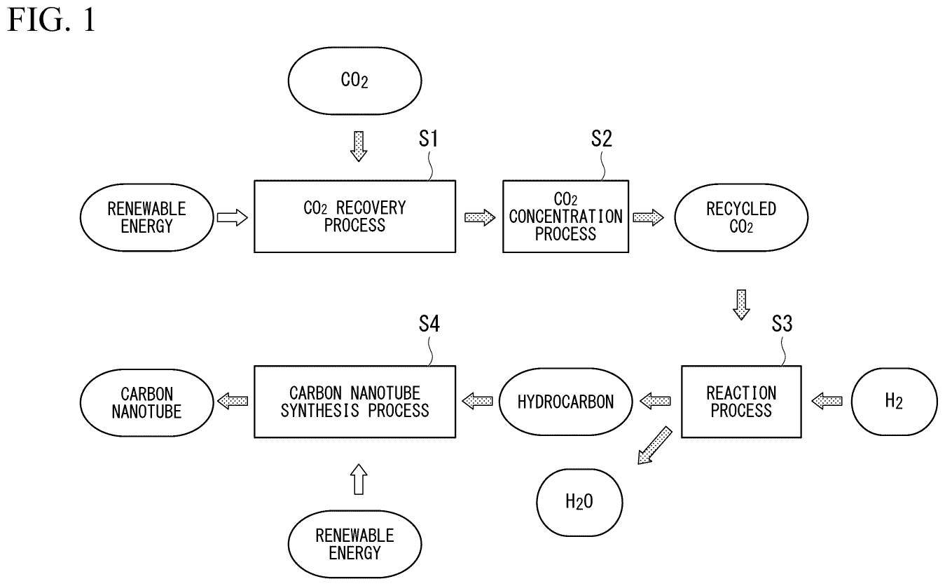

FIG. 1 is a conceptual diagram of a carbon nanotube production method according to Embodiment 1. The carbon nanotube production method according to Embodiment 1 includes, as shown in FIG. 1, a carbon dioxide recovery process S1 (recovery step), a carbon dioxide concentration process S2, a reaction process S3 (hydrocarbon generation step), and a carbon nanotube synthesis process S4 (carbon nanotube generation step).

The carbon dioxide recovery process S1 is a step of recovering carbon dioxide (CO₂) from the air by using energy. For example, in the carbon dioxide recovery process S1, the carbon dioxide is recovered from the air by using renewable energy. The renewable energy is, for example, electric power obtained by using solar light, wind power, geothermal power, small and medium hydro power, or biomass. The carbon nanotube production method according to the present embodiment 1 may include a step of generating the renewable energy. By using the renewable energy in the carbon dioxide recovery process S1, it is possible to reduce the discharge amount of greenhouse gases.

In the carbon dioxide recovery process S1, for example, the carbon dioxide is recovered from the air. The air is, for example, outside air. The air may be indoor air. The air may be an exhaust gas from a factory or the like. That is, the gas from which the carbon dioxide is recovered in the carbon dioxide recovery process S1 is not limited to the ambient air.

In addition, in the carbon dioxide recovery process S1, for example, a gas containing the carbon dioxide is separated from the air transported using a fan driven by the renewable energy. For such separation of the gas containing the carbon dioxide, for example, any one or a plurality of separation methods such as an adsorption separation method, a membrane separation method, a cooling separation method, a centrifugal separation method, a gravity separation method, or a gas-liquid separation method are employed.

The adsorption separation method is, for example, a method of performing separation by adsorbing a specific component onto an adsorbent or an adsorption liquid. Examples of the adsorbent include silica gel, zeolite, and activated carbon. Specifically, by adsorbing the component containing the carbon dioxide onto the adsorbent, this component can be separated from other components. The adsorbent may be in a granular form, a powdery form, or the like. The granular form is, for example, a bead form (spherical form) or a pellet form (cylindrical form). In a case in which the adsorbent in the powdery form is used, the adsorbent may be supported on a surface of a base material. The base material may have, for example, a honeycomb shape.

In the adsorption separation method, the carbon dioxide is separated from the adsorbent. For example, the carbon dioxide is separated from the adsorbent by heating the adsorbent. In addition, the carbon dioxide may be separated from the adsorbent by placing the adsorbent under a reduced pressure.

The membrane separation method is, for example, a method of separating a specific component from other components by using a permeable membrane through which low-molecular-weight components can pass. Specifically, for example, a component containing hydrogen (H₂) can be separated from the component including the carbon dioxide by using a palladium permeable membrane.

The cooling separation method is, for example, a method in which a specific component is liquefied by cooling to separate the specific component from other components (gas). Specifically, for example, a component containing water (H₂O) can be liquefied and separated from a gas containing the carbon dioxide.

The centrifugal separation method is, for example, a method in which a specific component (component containing water) is liquefied by cooling and is separated from other components (gas containing the carbon dioxide) by using a centrifugal force. The gravity separation method is, for example, a method in which a specific component (component containing water) is liquefied by cooling and is separated from other components (gas containing the carbon dioxide) by using a gravitational force. The gas-liquid separation method is, for example, a method in which a specific component (component containing water) is liquefied by cooling and separated from other components (gas containing the carbon dioxide) using gravitational force, centrifugal force, surface tension, or the like.

In the carbon dioxide concentration process S2, the concentration of the carbon dioxide (referred to as recycled carbon dioxide) recovered in the carbon dioxide recovery process S1 is increased. As in the carbon dioxide recovery process S1, in the recycled carbon dioxide concentration process S2, a concentration of the carbon dioxide is increased by employing any one or a plurality of separation methods such as the adsorption separation method, the membrane separation method, the cooling separation method, the centrifugal separation method, the gravity separation method, and the gas-liquid separation method. It should be noted that, in a case in which the concentration of the recycled carbon dioxide recovered in the carbon dioxide recovery process S1 is high, the carbon dioxide concentration process S2 may be omitted.

The reaction process S3 is a step of generating a hydrocarbon by using the carbon dioxide (recycled carbon dioxide) that is recovered in the carbon dioxide recovery process S1 and that is concentrated as necessary in the carbon dioxide concentration process S2. In the reaction process S3, water is generated in addition to the hydrocarbon.

In the carbon nanotube production method according to the present embodiment 1, in the reaction process S3, the hydrocarbon is generated from the carbon dioxide by using a Fischer-Tropsch reaction. More specifically, the hydrocarbon is synthesized from a mixed gas in which the recycled carbon dioxide and an externally supplied hydrogen are mixed, by using a catalyst. It should be noted that renewable energy can be used as the energy required in the reaction process S3.

Examples of the hydrocarbon generated in the reaction process S3 include propane, isobutane, dimethyl ether (DME), and acetylene. However, the type of the hydrocarbon is not particularly limited. The hydrocarbon generated in the reaction process S3 is, for example, a liquid. The hydrocarbon is generated from the carbon dioxide recovered in the carbon dioxide recovery process S1, and includes a carbon contained in the carbon dioxide recovered in the carbon dioxide recovery process S1.

In the carbon nanotube synthesis process S4, the carbon nanotube is generated by using the hydrocarbon generated in the reaction process S3 as a raw material. In the reaction process S3, the carbon nanotube is generated by using a chemical vapor deposition (CVD) method. Examples of the CVD method include a catalytic chemical vapor deposition (CCVD) method. The catalytic chemical vapor deposition method involves thermally decomposing the hydrocarbon, which serves as a carbon source, in a reaction furnace at a temperature of about 700° C. to 1000° C. in the presence of a catalytic metal, and then reacting the thermally decomposed carbon source with the catalytic metal. Examples of the catalytic chemical vapor deposition method include a method using methane as the carbon source (plasma-enhanced CCVD method). Examples of the catalytic chemical vapor deposition method also include a method (thermal CCVD method) using acetylene or ethylene as the carbon source. As the catalytic metal used in the catalytic chemical vapor deposition method, for example, iron, cobalt, or nickel is mainly used.

In addition, as the CVD method, a water-assisted-CCVD method (super-growth method) may be used. The super-growth method is an innovative carbon nanotube synthesis technology with a production efficiency approximately 1,000 times greater than that of a general CVD method. The super-growth method is a type of the thermal CCVD method, and is a generation method characterized by the addition of extremely low-concentration water along with the carbon source during the carbon nanotube generation step.

The carbon nanotube generated in the carbon nanotube synthesis process S4 is used as, for example, a raw material for a composite material. By using the composite material containing the carbon nanotube, a component (for example, a heat exchanger) of a heat pump device can be produced. That is, the heat pump device stores the carbon nanotube generated in the carbon nanotube synthesis process S4.

The carbon nanotube generated in the carbon nanotube synthesis process S4 is formed of the carbon contained in the carbon dioxide (recycled carbon dioxide) recovered in the carbon dioxide recovery process S1. Therefore, the heat pump device that stores the carbon nanotube stores at least the carbon contained in the carbon dioxide recovered in the carbon dioxide recovery process S1.

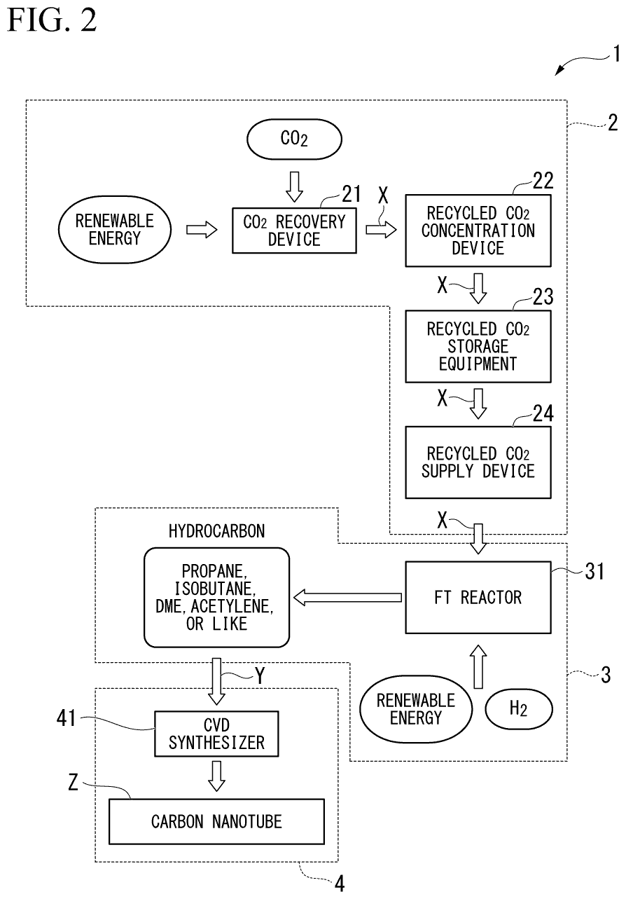

FIG. 2 is a block diagram showing a carbon nanotube production system 1 that produces the carbon nanotube by using the carbon nanotube production method according to Embodiment 1. As shown in FIG. 2, the carbon nanotube production system 1 according to Embodiment 1 includes a carbon dioxide recovery system 2, a hydrocarbon generation system 3, and a carbon nanotube generation system 4 (carbon nanotube generation device).

The carbon dioxide recovery system 2 recovers the carbon dioxide from the air by using the energy. For example, the carbon dioxide recovery system 2 recovers the carbon dioxide from the air by using renewable energy. The carbon nanotube production system 1 according to the present embodiment 1 may include a device that generates renewable energy. In a case in which the carbon dioxide recovery system 2 uses the renewable energy, it is possible to reduce the discharge amount of greenhouse gases.

As shown in FIG. 2, the carbon dioxide recovery system 2 includes a carbon dioxide recovery device 21, a recycled carbon dioxide concentration device 22, recycled carbon dioxide storage equipment 23, and a recycled carbon dioxide supply device 24.

The carbon dioxide recovery device 21 performs the carbon dioxide recovery process S1. As shown in FIG. 2, the carbon dioxide recovery device 21 is supplied with the air containing the carbon dioxide and the renewable energy. The carbon dioxide recovery device 21 uses the renewable energy to recover the carbon dioxide from the air. For example, the carbon dioxide recovery device 21 includes a fan operated by the renewable energy. Further, the carbon dioxide recovery device 21 includes a separation device that separates a gas containing the carbon dioxide from the air transported using the fan. For example, the separation device employs any one or a plurality of separation methods such as adsorption separation, membrane separation, cooling separation, centrifugal separation, gravity separation, or gas-liquid separation.

The recycled carbon dioxide concentration device 22 performs the carbon dioxide concentration process S2. The recycled carbon dioxide concentration device 22 increases a concentration of the carbon dioxide (recycled carbon dioxide X) recovered by the carbon dioxide recovery device 21. As in the carbon dioxide recovery device 21, the recycled carbon dioxide concentration device 22 increases a concentration of the carbon dioxide by employing any one or a plurality of separation methods such as the adsorption separation, the membrane separation, the cooling separation, the centrifugal separation, the gravity separation, or the gas-liquid separation. It should be noted that, in a case in which the concentration of the recycled carbon dioxide X recovered by the carbon dioxide recovery device 21 is high, the recycled carbon dioxide concentration device 22 may be omitted.

The recycled carbon dioxide storage equipment 23 is equipment that temporarily stores the recycled carbon dioxide X. The recycled carbon dioxide storage equipment 23 includes, for example, a storage tank. For example, the recycled carbon dioxide X stored in the recycled carbon dioxide storage equipment 23 is stored in a liquefied state by being cooled. The volume of the recycled carbon dioxide X can be reduced by liquefying the recycled carbon dioxide X. The recycled carbon dioxide X can be carried in and out of the recycled carbon dioxide storage equipment 23 by using a pipe or the like. In addition, the recycled carbon dioxide X may be carried in and out of the recycled carbon dioxide storage equipment 23 by using a transport container.

The recycled carbon dioxide supply device 24 supplies the recycled carbon dioxide X stored in the recycled carbon dioxide storage equipment 23 to the hydrocarbon generation system 3. It should be noted that the recycled carbon dioxide supply device 24 may supply the recycled carbon dioxide X to a container, such as a cylinder, that temporarily stores the recycled carbon dioxide X before the recycled carbon dioxide X is supplied to the hydrocarbon generation system 3.

The hydrocarbon generation system 3 performs the reaction process S3. The hydrocarbon generation system 3 generates hydrocarbon Y by using the carbon dioxide (recycled carbon dioxide X) recovered by carbon dioxide recovery system 2. In the present embodiment 1, the hydrocarbon generation system 3 includes an FT reactor 31.

In the FT reactor 31, the hydrocarbon Y is generated from the carbon dioxide by using a Fischer-Tropsch reaction. In the FT reactor 31, the hydrocarbon Y is synthesized from a mixed gas in which the recycled carbon dioxide and externally supplied hydrogen are mixed, by using a catalyst. It should be noted that renewable energy can be used as the energy required in the FT reactor 31.

The hydrocarbon Y generated in the FT reactor 31 is, for example, a liquid. Therefore, the hydrocarbon Y generated in the FT reactor 31 can be used as, for example, a heat medium of the heat pump device. By supplying the hydrocarbon Y as the heat medium of the heat pump device, the heat pump device stores the hydrocarbon Y. That is, the heat pump device that stores the hydrocarbon Y stores at least the carbon contained in the carbon dioxide recovered by the carbon dioxide recovery system 2.

The carbon nanotube generation system 4 performs the carbon nanotube synthesis process S4. The carbon nanotube generation system 4 generates a carbon nanotube Z by using the hydrocarbon Y generated by the hydrocarbon generation system 3 as a raw material. In the present embodiment 1, the carbon nanotube generation system 4 includes a CVD synthesizer 41. The CVD synthesizer 41 generates the carbon nanotube Z by using a chemical vapor deposition method.

In the carbon nanotube production system 1 according to the present embodiment 1, the carbon dioxide recovery system 2 recovers the carbon dioxide from the air by using the energy. Specifically, the carbon dioxide is recovered from the air by the carbon dioxide recovery device 21 by using the renewable energy or the like. The carbon dioxide (recycled carbon dioxide X) recovered by the carbon dioxide recovery device 21 is concentrated by the recycled carbon dioxide concentration device 22. In the recycled carbon dioxide concentration device 22, the concentration of the recycled carbon dioxide is increased.

The recycled carbon dioxide X concentrated by the recycled carbon dioxide concentration device 22 is temporarily stored in the recycled carbon dioxide storage equipment 23. The recycled carbon dioxide supply device 24 supplies the recycled carbon dioxide X stored in the recycled carbon dioxide storage equipment 23 to the hydrocarbon generation system 3.

The recycled carbon dioxide X is supplied to the hydrocarbon generation system 3, and the hydrocarbon generation system 3 generates the hydrocarbon Y. The hydrocarbon Y generated in the hydrocarbon generation system 3 is used as a raw material of the carbon nanotube Z. The hydrocarbon Y is supplied to the carbon nanotube generation system 4, and the carbon nanotube generation system 4 generates the carbon nanotube Z.

The carbon nanotube production method according to the present embodiment 1 described above includes the carbon dioxide recovery process S1, the reaction process S3, and the carbon nanotube synthesis process S4. The carbon dioxide recovery process S1 is a step of recovering the carbon dioxide from the air by using the energy. The reaction process S3 is a step of generating the hydrocarbon Y by using the recovered carbon dioxide (recycled carbon dioxide X). The carbon nanotube synthesis process S4 is a step of generating the carbon nanotube Z by using the hydrocarbon Y as a raw material.

With the carbon nanotube production method according to the present embodiment 1, the recovered carbon dioxide can be effectively used as the carbon nanotube Z. For example, by using the carbon nanotube Z as a raw material of the composite material and incorporating the carbon nanotube Z into a device such as a heat pump device, the carbon can be fixed to the device such as the heat pump device. Therefore, with the carbon nanotube production method according to the present embodiment 1, the intended use of the recovered carbon dioxide can be expanded and the carbon dioxide released into the air can be reduced.

In addition, the carbon nanotube production system 1 according to the present embodiment described above includes the carbon dioxide recovery system 2, the hydrocarbon generation system 3, and the carbon nanotube generation system 4. The carbon dioxide recovery system 2 recovers the carbon dioxide from the air by using the energy. The hydrocarbon generation system 3 generates the hydrocarbon Y by using the recycled carbon dioxide X recovered by the carbon dioxide recovery system 2. The carbon nanotube generation system 4 generates a carbon nanotube Z by using the hydrocarbon Y as a raw material.

With the carbon nanotube production system 1 according to the present embodiment 1, the recovered carbon dioxide can be effectively used as the carbon nanotube Z. For example, by using the carbon nanotube Z as a raw material of the composite material and incorporating the carbon nanotube Z into a device such as a heat pump device, the carbon can be fixed to the device such as the heat pump device. Therefore, with the carbon nanotube production system 1 according to the present embodiment 1, the intended use of the recovered carbon dioxide can be expanded and the carbon dioxide released into the air can be reduced.

### Embodiment 2

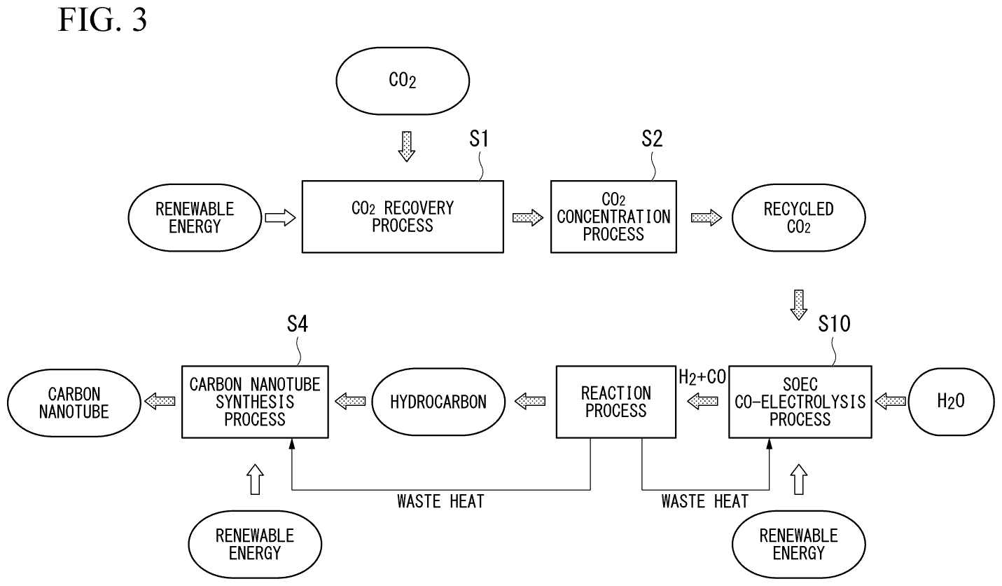

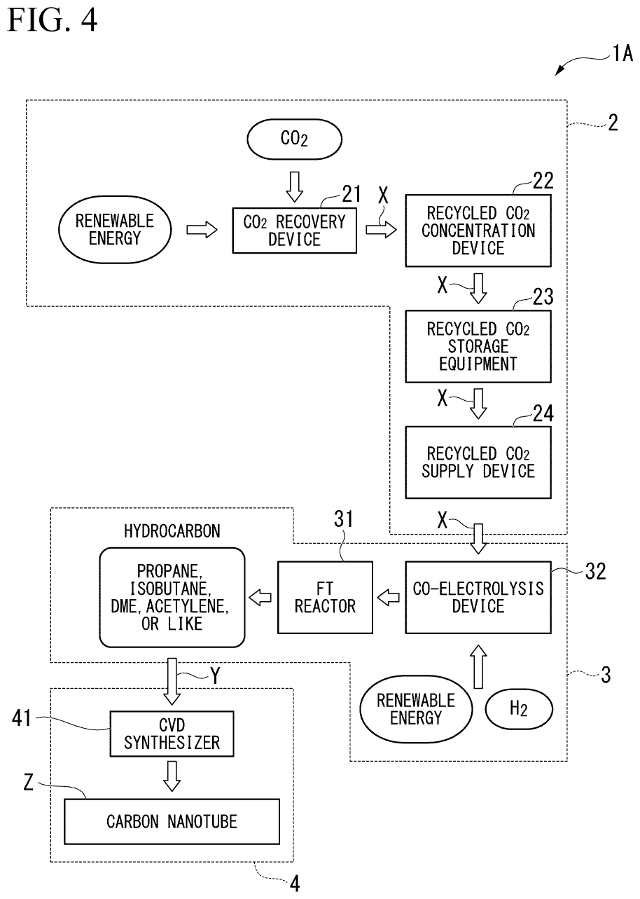

Next, Embodiment 2 of the present disclosure will be described with reference to FIGS. 3 and 4. It should be noted that, in the description of the present embodiment 2, the description of the same parts as those in the description of Embodiment 1 will be omitted or simplified.

FIG. 3 is a conceptual diagram of a carbon nanotube production method according to Embodiment 2. As shown in FIG. 3, in the carbon nanotube production method according to the present embodiment 2, an SOEC co-electrolysis process S10 (co-electrolysis step) is performed between the carbon dioxide concentration process S2 and the reaction process S3. In the present embodiment, the hydrocarbon Y is generated in the reaction process S3 and the SOEC co-electrolysis process S10. That is, the hydrocarbon generation step includes the reaction process S3 (reaction step) and the SOEC co-electrolysis process S10.

In the SOEC co-electrolysis process S10, a mixed gas containing carbon monoxide (CO) and hydrogen is obtained from the carbon dioxide and the water via the co-electrolysis. In the SOEC co-electrolysis process S10, a solid oxide electrolysis cell (SOEC), including a cathode electrode and an anode electrode, is used. For example, in the solid oxide electrolysis cell, solid oxide with oxygen ion conductivity is used. As the electrolyte, zirconia-based oxides or the like is used. In the SOEC co-electrolysis process S10, the supplied water (or water and carbon dioxide) is supplied to the cathode electrode of the solid oxide electrolysis cell. It is desirable that the water used in the co-electrolysis in the solid oxide electrolysis cell is steam. In addition, in the SOEC co-electrolysis process S10, a recovery gas containing the carbon dioxide is supplied to the cathode electrode of the solid oxide electrolysis cell.

The solid oxide electrolysis cell may be heated in the SOEC co-electrolysis process S10. By heating the solid oxide electrolysis cell, a temperature inside the solid oxide electrolysis cell can be adjusted to a temperature suitable for the co-electrolysis reaction. The ratio of carbon dioxide to water that are supplied to the solid oxide electrolysis cell can be determined by the ratio of the components (carbon monoxide and hydrogen) of the target mixed gas.

It should be noted that the device for obtaining the carbon monoxide and the hydrogen is not limited to the SOEC co-electrolysis process. For example, an electrolysis process can also be used in which a step of electrolyzing the carbon dioxide to obtain the carbon monoxide and a step of electrolyzing the water to obtain the hydrogen are independently performed.

In a case in which the SOEC co-electrolysis process S10 is performed, in the reaction process S3, the hydrocarbon Y is generated from the carbon monoxide. In the reaction process S3, the hydrocarbon Y is synthesized from the mixed gas in which the hydrogen and the carbon monoxide generated in the SOEC co-electrolysis process S10 are mixed, by using the catalyst.

In addition, in the present embodiment 2, as shown in FIG. 3, a part of waste heat obtained in the reaction process S3 may be used in the SOEC co-electrolysis process S10 and the carbon nanotube synthesis process S4. It should be noted that a part of the waste heat obtained in the reaction process S3 may be used in any one of the SOEC co-electrolysis process S10 and the carbon nanotube synthesis process S4.

FIG. 4 is a block diagram showing a carbon nanotube production system 1A that produces the carbon nanotube by using the carbon nanotube production method according to Embodiment 2. As shown in FIG. 4, in the carbon nanotube production system 1A according to Embodiment 2, the hydrocarbon generation system 3 performs the SOEC co-electrolysis process S10 and the reaction process S3. In the present embodiment 2, the hydrocarbon generation system 3 includes a co-electrolysis device 32 and the FT reactor 31.

The co-electrolysis device 32 is supplied with the recycled carbon dioxide X and generates carbon monoxide and hydrogen from the recycled carbon dioxide X. In this case, as shown in FIG. 4, the co-electrolysis device 32 may generate the carbon monoxide and the hydrogen by using the waste heat from the FT reactor 31. The use of the waste heat of the FT reactor 31 means that the heat generated in the FT reactor 31 is supplied via the heat medium, and the supplied heat is used via the heat medium. The FT reactor 31 generates the hydrocarbon Y from the carbon monoxide and the hydrogen generated by the co-electrolysis device 32.

In addition, in the present embodiment 2, as shown in FIG. 4, the CVD synthesizer 41 may generate the carbon nanotube Z by using the waste heat of the FT reactor 31.

The carbon nanotube production method according to the present embodiment 2 described above includes the SOEC co-electrolysis process S10 and the reaction process S3. The SOEC co-electrolysis process S10 is a step of generating the carbon monoxide from the recycled carbon dioxide X by using the solid oxide. The reaction process S3 is a step of generating the hydrocarbon Y from the carbon monoxide by using the Fischer-Tropsch method.

In the carbon nanotube production method according to the present embodiment 2 as well, the hydrocarbon Y is generated, and the carbon nanotube Z is generated from the hydrocarbon Y. Therefore, with the carbon nanotube production method according to the present embodiment 2, the recovered carbon dioxide can be effectively used as the carbon nanotube Z.

In addition, in the carbon nanotube production method according to the present embodiment 2, at least a part of the waste heat of the reaction process S3 may be used in at least the SOEC co-electrolysis process S10 and/or the carbon nanotube synthesis process S4. In a case in which at least a part of the waste heat of the reaction process S3 is used in the SOEC co-electrolysis process S10, the hydrocarbon conversion efficiency (generation amount of hydrocarbon relative to input energy) can be increased. In addition, in a case in which at least a part of the waste heat of the reaction process S3 is used in the carbon nanotube synthesis process S4, the carbon nanotube conversion efficiency (generation amount of carbon nanotube relative to input energy) can be increased.

In addition, in the carbon nanotube production system 1A according to the present embodiment 2, the hydrocarbon generation system 3 includes the co-electrolysis device 32 and the FT reactor 31. The co-electrolysis device 32 generates the carbon monoxide from the recycled carbon dioxide X by using the solid oxide. In the FT reactor 31, the hydrocarbon Y is generated from the carbon monoxide by using the Fischer-Tropsch method.

In the carbon nanotube production system 1A according to the present embodiment 2 as well, the hydrocarbon Y is generated, and the carbon nanotube Z is generated from the hydrocarbon Y. Therefore, with the carbon nanotube production system 1A according to the present embodiment 2, the recovered carbon dioxide can be effectively used as the carbon nanotube Z.

### Embodiment 3

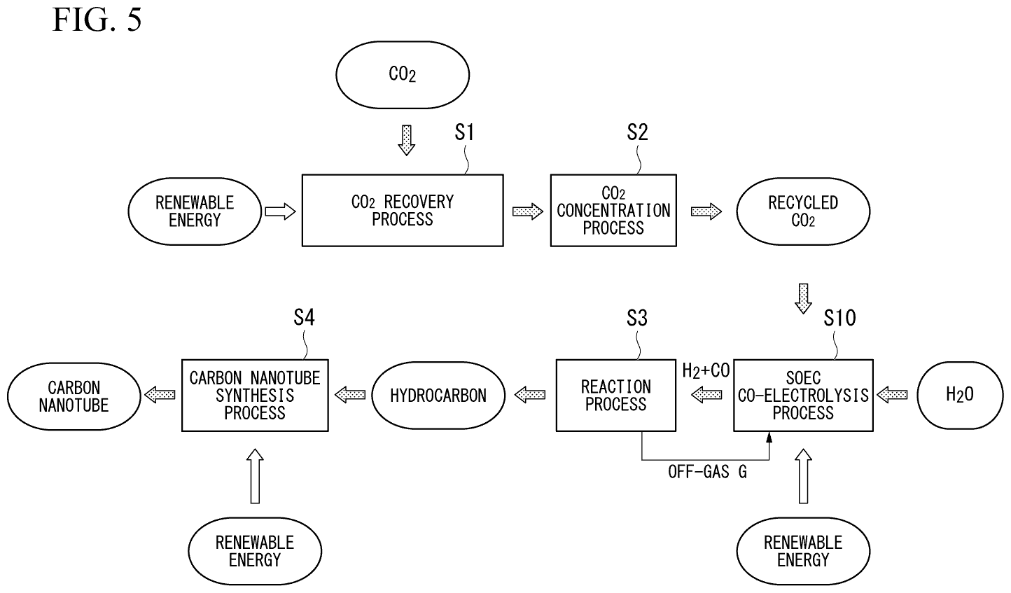

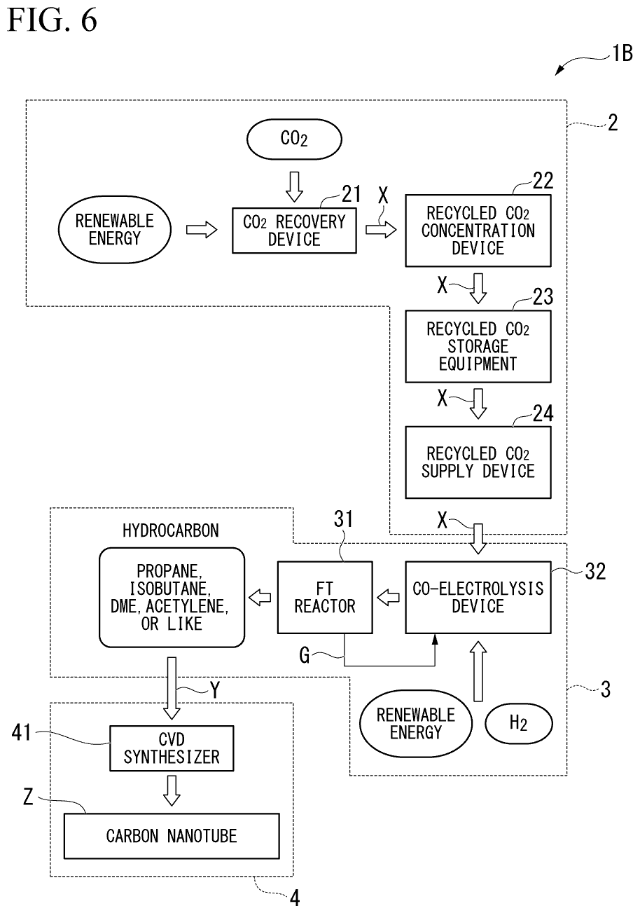

Next, Embodiment 3 of the present disclosure will be described with reference to FIGS. 5 and 6. It should be noted that, in the description of the present embodiment 3, the description of the same parts as those in the description of Embodiment 1 or Embodiment 2 will be omitted or simplified.

FIG. 5 is a conceptual diagram of a carbon nanotube production method according to Embodiment 3. As shown in FIG. 5, in the carbon nanotube production method according to the present embodiment 3, an off-gas G of hydrogen generated in the reaction process S3 is used in the SOEC co-electrolysis process S10.

FIG. 6 is a block diagram showing a carbon nanotube production system 1B that produces the carbon nanotube by using the carbon nanotube production method according to Embodiment 3. As shown in FIG. 6, in the carbon nanotube production system 1B according to Embodiment 3, the off-gas G including unreacted hydrogen is supplied from the FT reactor 31 to the co-electrolysis device 32.

In the carbon nanotube production method according to the present embodiment 3 described above, the off-gas G of the hydrogen generated in the reaction process S3 is used in the SOEC co-electrolysis process S10. With the carbon nanotube production method according to the present embodiment 3, the off-gas G of the hydrogen generated in the reaction process S3 can be effectively used, and the hydrocarbon conversion efficiency can be further increased. In addition, the waste heat of the reaction process S3 is supplied to the SOEC co-electrolysis process S10 along with the off-gas G, and thus the waste heat of the reaction process S3 can be effectively used.

### Embodiment 4

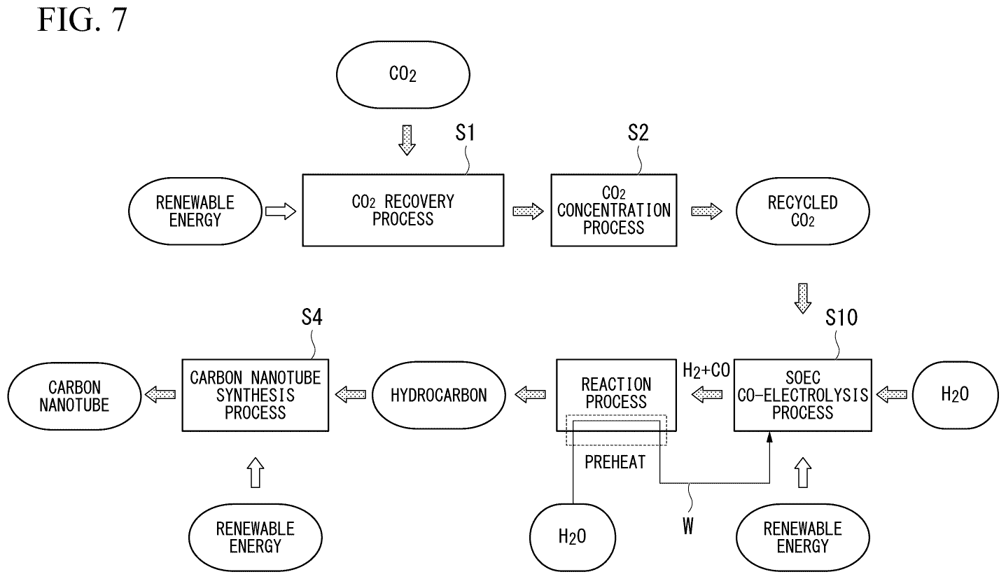

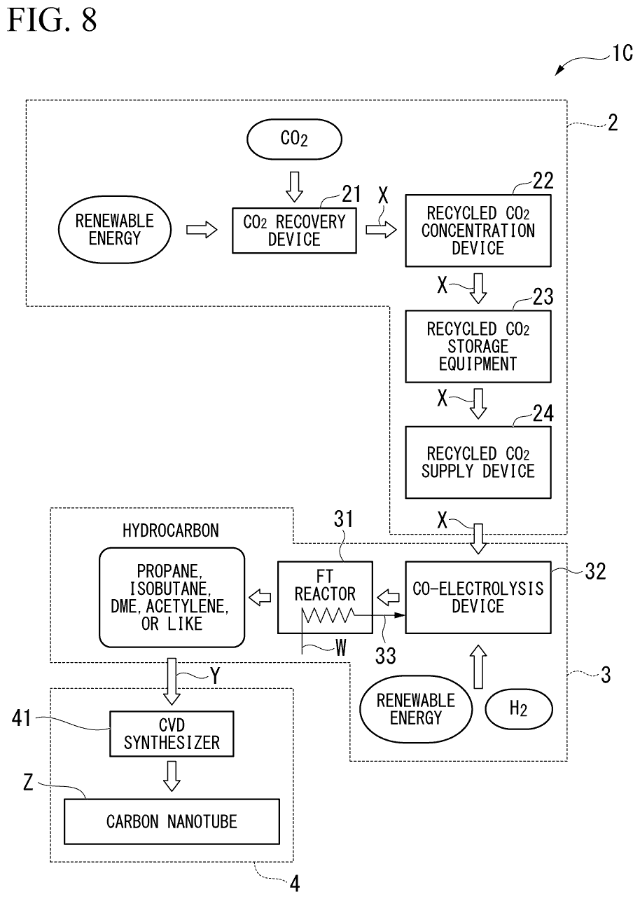

Next, Embodiment 4 of the present disclosure will be described with reference to FIGS. 7 and 8. It should be noted that, in the description of the present embodiment 4, the description of the same parts as those in the description of Embodiment 1 or Embodiment 2 will be omitted or simplified.

FIG. 7 is a conceptual diagram of a carbon nanotube production method according to Embodiment 4. As shown in FIG. 7, in the carbon nanotube production method according to the present embodiment 3, the water W used in the SOEC co-electrolysis process S10 is preheated in the reaction process S3. For example, the water W used in the SOEC co-electrolysis process S10 is heated by using the heat generated in the reaction process S3.

FIG. 8 is a block diagram showing a carbon nanotube production system 1C that produces the carbon nanotube by using the carbon nanotube production method according to Embodiment 4. As shown in FIG. 8, in the carbon nanotube production system 1C according to the present embodiment 4, a pipe 33 for the water W, which is connected to the co-electrolysis device 32, is provided to pass through the FT reactor 31. In the carbon nanotube production system 1C according to the present embodiment 4, the water W flowing through the pipe 33 is preheated in the FT reactor 31 and then supplied to the co-electrolysis device 32.

In the carbon nanotube production method according to the present embodiment 4 as described above, the water W used in the SOEC co-electrolysis process S10 is preheated in the reaction process S3. Therefore, the heat of the reaction process S3 can be effectively used in the SOEC co-electrolysis process S10, and thus the improvement in the hydrocarbon conversion efficiency is expected. Further, in the carbon nanotube production method according to the present embodiment 4, the FT reactor 31 can be cooled with the water W used in the SOEC co-electrolysis process S10.

### Embodiment 5

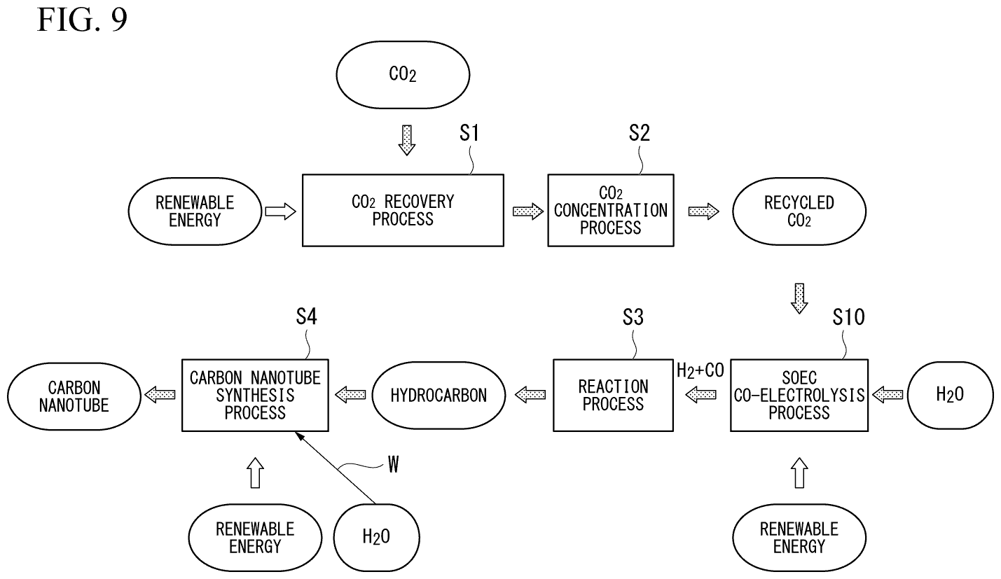

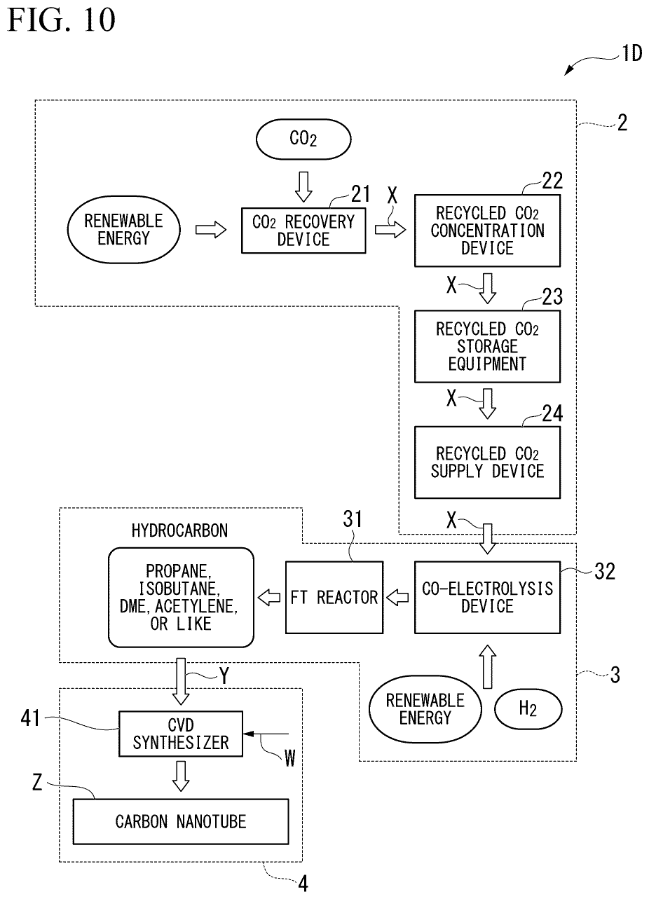

Next, Embodiment 5 of the present disclosure will be described with reference to FIGS. 9 and 10. It should be noted that, in the description of the present embodiment 5, the description of the same parts as those in the description of Embodiment 1 or Embodiment 2 will be omitted or simplified.

FIG. 9 is a conceptual diagram of a carbon nanotube production method according to Embodiment 5. As shown in FIG. 9, in the carbon nanotube production method according to the present embodiment 5, the water W is used in the carbon nanotube synthesis process S4. In the carbon nanotube production method according to the present embodiment 5, for example, the carbon nanotube Z is generated by using a super-growth method.

FIG. 10 is a block diagram showing a carbon nanotube production system 1D that produces the carbon nanotube by using the carbon nanotube production method according to Embodiment 5. As shown in FIG. 10, in the carbon nanotube production system 1D according to the present embodiment 5, the water W is supplied to the CVD synthesizer 41.

In the carbon nanotube production method according to the present embodiment 5 as described above, for example, the carbon nanotube Z can be generated by using the super-growth method. Therefore, it is possible to significantly improve the production efficiency of the carbon nanotube Z.

### Embodiment 6

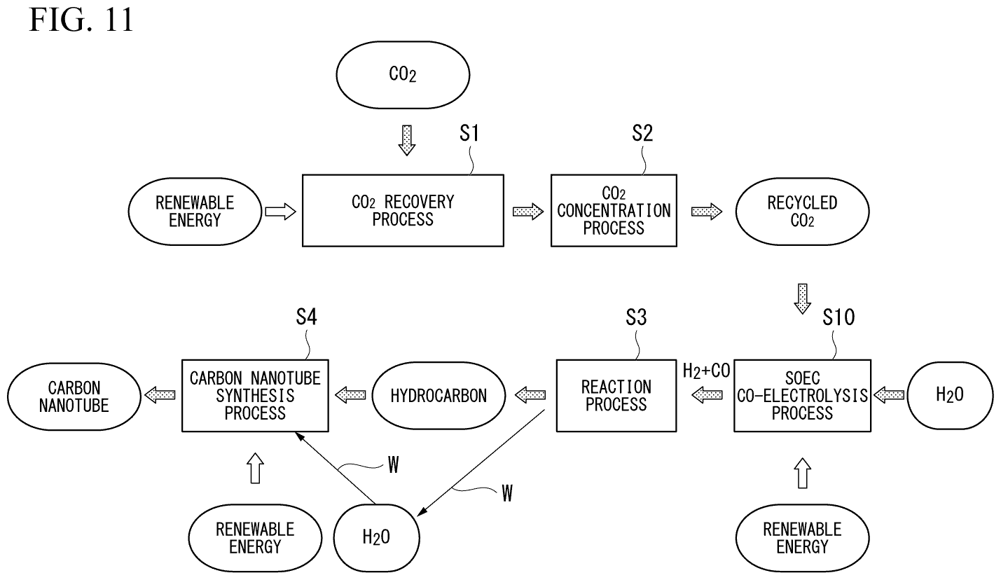

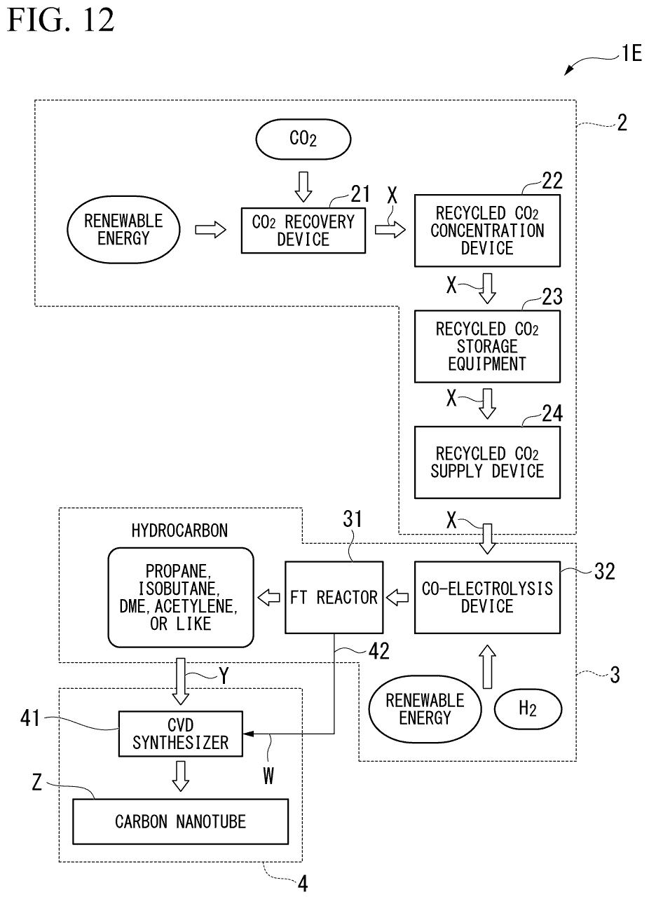

Next, Embodiment 6 of the present disclosure will be described with reference to FIGS. 11 and 12. It should be noted that, in the description of the present embodiment 6, the description of the same parts as those in the description of Embodiment 1, Embodiment 2, or Embodiment 5 will be omitted or simplified.

FIG. 11 is a conceptual diagram of a carbon nanotube production method according to Embodiment 6. As shown in FIG. 11, in the carbon nanotube production method according to the present embodiment 6, the water W generated in the reaction process S3 is supplied to the carbon nanotube synthesis process S4.

FIG. 12 is a block diagram showing a carbon nanotube production system 1E that produces the carbon nanotube by using the carbon nanotube production method according to Embodiment 6. As shown in FIG. 12, in the carbon nanotube production system 1E according to the present embodiment 6, the CVD synthesizer 41 and the FT reactor 31 are connected via a pipe 42. The water W is supplied to the CVD synthesizer 41 from the FT reactor 31.

In the carbon nanotube production method according to the present embodiment 5 as described above, for example, the carbon nanotube Z can be generated by using the super-growth method. Therefore, it is possible to significantly improve the production efficiency of the carbon nanotube Z. Further, since the water W generated in the reaction process S3 is supplied to the carbon nanotube synthesis process S4, the water W generated in the reaction process S3 can be effectively used. In addition, the waste heat of the reaction process S3 is supplied to the carbon nanotube synthesis process S4 along with the water W, and thus the waste heat of the reaction process S3 can be effectively used.

### Embodiment 7

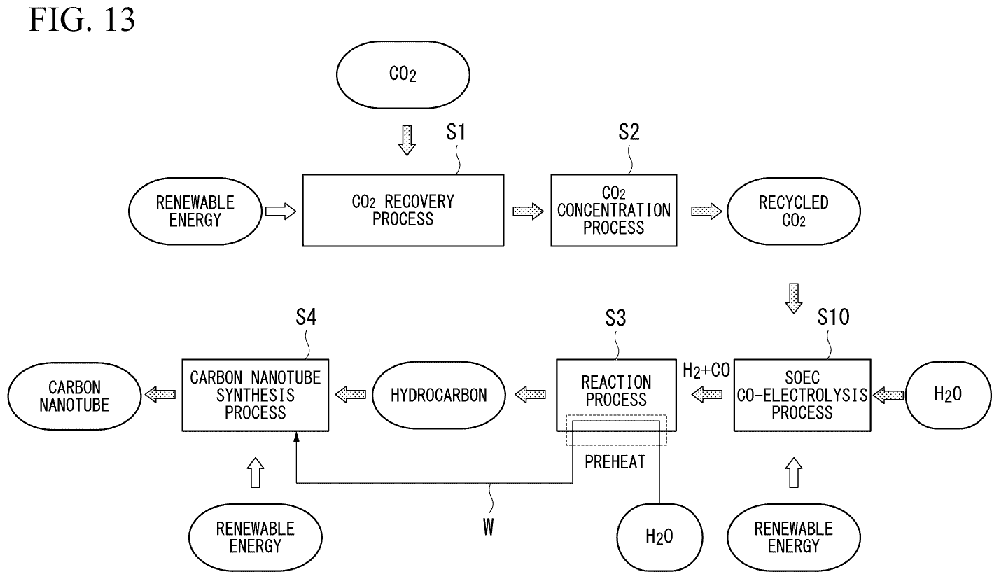

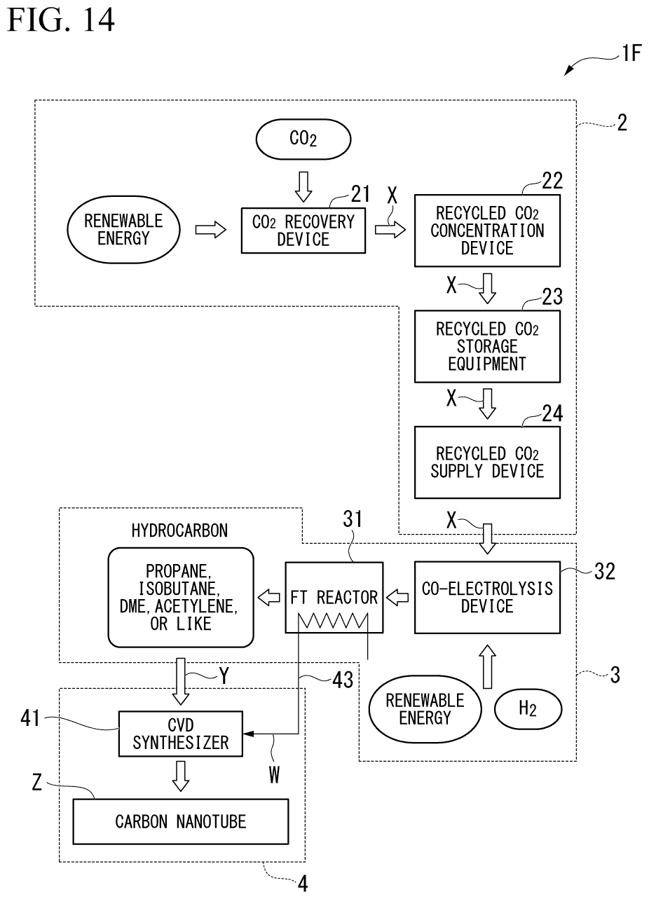

Next, Embodiment 7 of the present disclosure will be described with reference to FIGS. 13 and 14. It should be noted that, in the description of the present embodiment 7, the description of the same parts as those in the description of Embodiment 1, Embodiment 2, or Embodiment 5 will be omitted or simplified.

FIG. 13 is a conceptual diagram of a carbon nanotube production method according to Embodiment 7. As shown in FIG. 13, in the carbon nanotube production method according to the present embodiment 7, the water W used in the carbon nanotube synthesis process S4 is preheated in the reaction process S3. For example, the water W used in the carbon nanotube synthesis process S4 is heated by using the heat generated in the reaction process S3.

FIG. 14 is a block diagram showing a carbon nanotube production system 1F that produces the carbon nanotube by using the carbon nanotube production method according to Embodiment 7. As shown in FIG. 14, in the carbon nanotube production system 1F according to the present embodiment 7, a pipe 42 for the water W, which is connected to the CVD synthesizer 41, is provided to pass through the FT reactor 31. In the carbon nanotube production system 1F according to the present embodiment 7, the water flowing through the pipe 42 is preheated in the FT reactor 31 and then supplied to the CVD synthesizer 41.

In the carbon nanotube production method according to the present embodiment 7 described above, the water used in the carbon nanotube synthesis process S4 is preheated in the reaction process S3. Therefore, the heat of the reaction process S3 can be effectively used in the carbon nanotube synthesis process S4, and thus the improvement in the productivity of carbon nanotube is expected. Further, in the carbon nanotube production method according to the present embodiment 7, the FT reactor 31 can be cooled with the water used in the carbon nanotube synthesis process S4.

### Embodiment 8

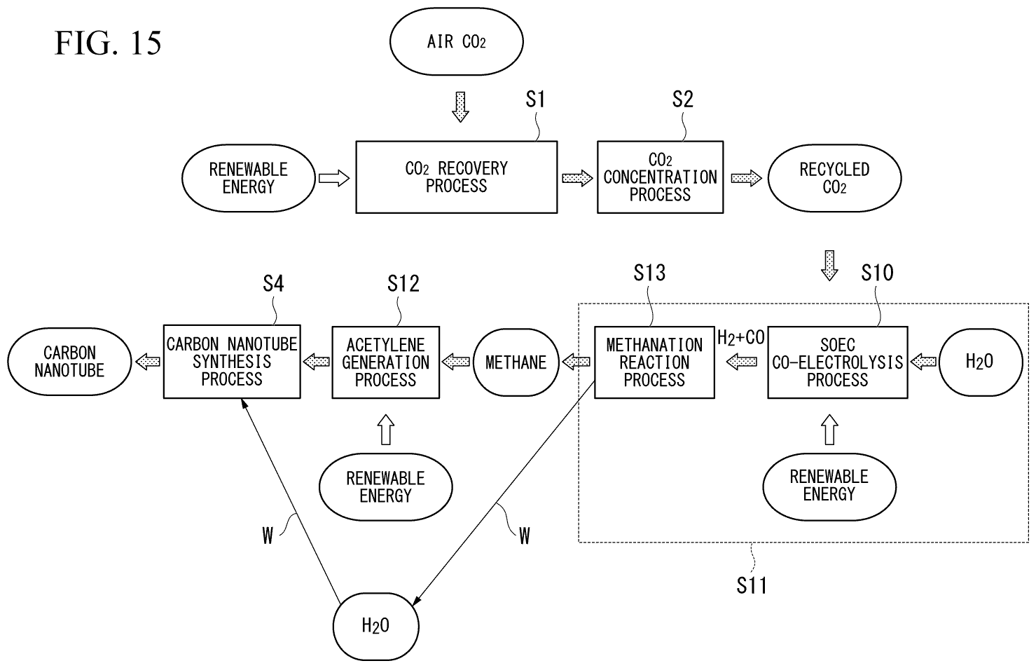

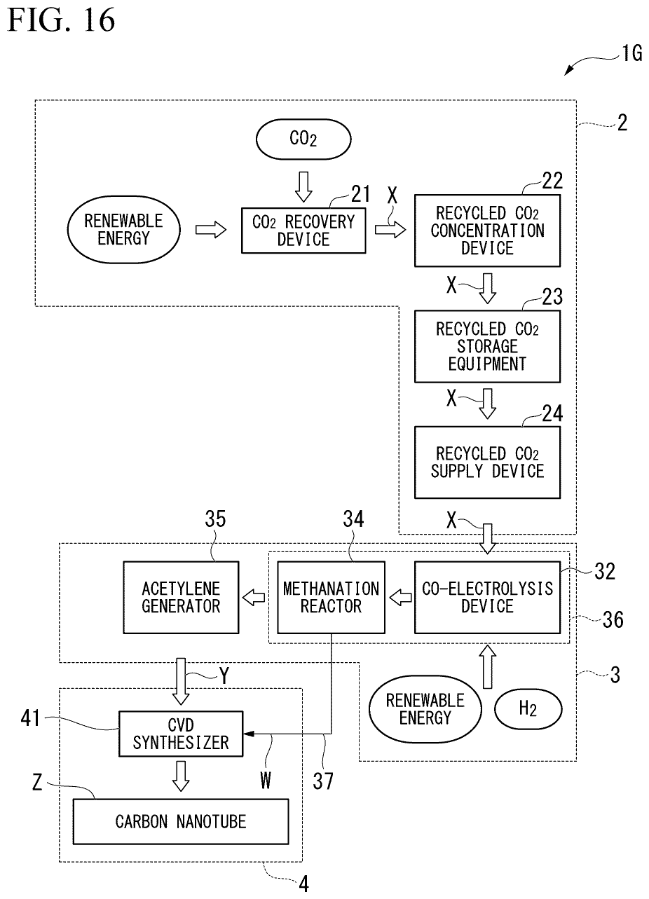

Next, Embodiment 8 of the present disclosure will be described with reference to FIGS. 15 and 16. It should be noted that, in the description of the present embodiment 8, the description of the same parts as those in the description of Embodiment 1 or Embodiment 2 will be omitted or simplified.

FIG. 15 is a conceptual diagram of a carbon nanotube production method according to Embodiment 8. As shown in FIG. 15, the carbon nanotube production method according to the present embodiment 8 includes a methane generation process S11 (methane generation step) and an acetylene generation process S12 (acetylene generation step).

In the present embodiment 8, the methane generation process S11 includes the SOEC co-electrolysis process S10. In addition, the methane generation process S11 includes a methanation reaction process S13 of generating methane from the carbon monoxide generated in the SOEC co-electrolysis process S10.

In the acetylene generation process S12, acetylene (hydrocarbon) is generated from the methane generated in the methane generation process S11. In the acetylene generation process S12, for example, acetylene is generated from the methane by using the renewable energy.

In addition, in the carbon nanotube synthesis process S4, the carbon nanotube Z is generated by using the acetylene as a raw material. Further, the water W generated in the methanation reaction process S13 is supplied to the carbon nanotube synthesis process S4.

In the present embodiment 8, the acetylene, which is the hydrocarbon, is generated in the methane generation process S11 and the acetylene generation process S12. That is, the hydrocarbon generation step includes the methane generation process S11 and the acetylene generation process S12.

FIG. 16 is a block diagram showing a carbon nanotube production system 1G that produces the carbon nanotube by using the carbon nanotube production method according to Embodiment 8. As shown in FIG. 16, in the carbon nanotube production system 1G according to the present embodiment 8, the hydrocarbon generation system 3 includes the co-electrolysis device 32, a methanation reactor 34, and an acetylene generator 35 (acetylene generation device).

The methanation reactor 34 performs the methanation reaction process S13. The methanation reactor 34 generates the methane from the carbon monoxide generated in the SOEC co-electrolysis process S10. The acetylene generator 35 performs the acetylene generation process S12. The acetylene generator 35 generates the acetylene (hydrocarbon Y) from the methane generated in the methanation reactor 34.

In addition, the CVD synthesizer 41 (carbon nanotube generation system 4) and the methanation reactor 34 are connected via a pipe 37. The CVD synthesizer 41 can generate the carbon nanotube Z by using the water W supplied from the methanation reactor 34 via the pipe 37.

The carbon nanotube production method according to the present embodiment 8 includes the methane generation process S11 of generating the methane from the carbon dioxide. In addition, the carbon nanotube production method according to the present embodiment 8 includes the acetylene generation process S12 of generating the acetylene from the methane. In addition, in the carbon nanotube production method according to the present embodiment 8, the carbon nanotube synthesis process S4 generates the carbon nanotube Z by using the acetylene as a raw material.

In the carbon nanotube production method according to the present embodiment 8 as well, the hydrocarbon Y is generated, and the carbon nanotube Z is generated from the hydrocarbon Y. Therefore, with the carbon nanotube production method according to the present embodiment 8, the recovered carbon dioxide can be effectively used as the carbon nanotube Z.

In addition, in the carbon nanotube production system 1G according to the present embodiment 8, the hydrocarbon generation system 3 includes a methane generation device 36 and the acetylene generator 35. The methane generation device 36 includes the co-electrolysis device 32 and the methanation reactor 34. That is, the methane generation device 36 generates the methane from the recycled carbon dioxide X. The acetylene generator 35 generates the acetylene from the methane. In addition, in the carbon nanotube production system 1G according to the present embodiment 8, the carbon nanotube generation system 4 generates the carbon nanotube Z by using the acetylene as a raw material.

In the carbon nanotube production system 1G according to the present embodiment 8 as well, the hydrocarbon Y is generated, and the carbon nanotube Z is generated from the hydrocarbon Y. Therefore, with the carbon nanotube production system 1G according to the present embodiment 8, the recovered carbon dioxide can be effectively used as the carbon nanotube Z.

Hitherto, the preferred embodiments of the present disclosure have been described with reference to the accompanying drawings, but it goes saying without that the present disclosure is not limited to the above-described embodiments. Various shapes or combinations of the configuration members shown in the above-described embodiments are merely examples, and can be modified in various ways based on design requirements within a range not departing from the gist of the present disclosure. In addition, the above-described embodiments may be combined as appropriate.

For example, in the above-described embodiments, the carbon dioxide recovery process S1 and the reaction process S3 (hydrocarbon generation step) can be regarded as a method of producing the hydrocarbon Y. That is, the method of producing the hydrocarbon Y includes the carbon dioxide recovery process S1 and the reaction process S3 (hydrocarbon generation step). In the carbon dioxide recovery process S1, the carbon dioxide is recovered from the air by using the energy. In the reaction process S3, the hydrocarbon Y is generated by using the recovered carbon dioxide. With the method of producing the hydrocarbon Y, the recovered carbon dioxide can be effectively used as the hydrocarbon Y. Therefore, the intended use of the recovered carbon dioxide can be expanded, and the carbon dioxide released into the air can be reduced.

### REFERENCE SIGNS LIST

1, 1A, 1B, 1C, 1D, 1E, 1F, 1G: Carbon nanotube production system
2: Carbon dioxide recovery system
3: Hydrocarbon generation system
4: Carbon nanotube generation system (carbon nanotube generation device)
21: Carbon dioxide recovery device
22: Recycled carbon dioxide concentration device
23: Recycled carbon dioxide storage equipment
24: Recycled carbon dioxide supply device
31: FT reactor
32: Co-electrolysis device
34: Methanation reactor
35: Acetylene generator (acetylene generation device)
36: Methane generation device
41: CVD synthesizer
G: Off-gas
S1: Carbon dioxide recovery process (recovery step)
S2: Carbon dioxide concentration process
S3: Reaction process (hydrocarbon production process)
S4: Carbon nanotube synthesis process (carbon nanotube generation step)
S10: SOEC co-electrolysis process (co-electrolysis step)
S11: Methane generation process (methane generation step)
S12: Acetylene generation process (acetylene generation step)
S13: Methanation reaction process
W: Water
X: Recycled carbon dioxide
Y: Hydrocarbon
Z: Carbon nanotube

What is claimed is:

1. A carbon nanotube production method comprising:
a recovery step of recovering carbon dioxide from air by using energy;
a hydrocarbon generation step of generating a hydrocarbon by using the recovered carbon dioxide; and
a carbon nanotube generation step of generating a carbon nanotube by using the hydrocarbon as a raw material.

2. The carbon nanotube production method according to claim 1,
wherein the hydrocarbon generation step includes:
a co-electrolysis step of generating carbon monoxide from the carbon dioxide by using solid oxide; and
a reaction step of generating the hydrocarbon from the carbon monoxide by using a Fischer-Tropsch method.

3. The carbon nanotube production method according to claim 2,
wherein at least a part of waste heat obtained in the reaction step is used in at least the co-electrolysis step and/or the carbon nanotube generation step.

4. The carbon nanotube production method according to claim 2,
wherein an off-gas of hydrogen generated in the reaction step is used in the co-electrolysis step.

5. The carbon nanotube production method according to claim 2,
wherein water used in the co-electrolysis step is preheated in the reaction step.

6. The carbon nanotube production method according to claim 2,
wherein water used in the carbon nanotube generation step is preheated in the reaction step.

7. The carbon nanotube production method according to claim 2,
wherein water generated in the reaction step is used in the carbon nanotube generation step.

8. The carbon nanotube production method according to claim 1,
wherein the hydrocarbon generation step includes:
a methane generation step of generating methane from the carbon dioxide; and
an acetylene generation step of generating acetylene from the methane, and
in the carbon nanotube generation step, the carbon nanotube is generated by using the acetylene as a raw material.

9. The carbon nanotube production method according to claim 8,
wherein water generated in the methane generation step is used in the carbon nanotube generation step.

10. The carbon nanotube production method according to claim 1,
wherein, in the carbon nanotube generation step, the carbon nanotube is generated by using a chemical vapor deposition method in which water is added along with the hydrocarbon.

11. A carbon nanotube production system comprising:
a carbon dioxide recovery system configured to recover carbon dioxide from air by using energy;
a hydrocarbon generation system configured to generate a hydrocarbon by using the carbon dioxide recovered by the carbon dioxide recovery system; and
a carbon nanotube generation device configured to generate a carbon nanotube by using the hydrocarbon as a raw material.

12. The carbon nanotube production system according to claim 11,
wherein the hydrocarbon generation system includes:
a co-electrolysis device configured to generate carbon monoxide from the carbon dioxide by using solid oxide; and
a reactor configured to generate the hydrocarbon from the carbon monoxide by using a Fischer-Tropsch method.

13. The carbon nanotube production system according to claim 11,
wherein the hydrocarbon generation system includes:
a methane generation device configured to generate methane from the carbon dioxide; and
an acetylene generation device configured to generate acetylene from the methane, and
the carbon nanotube generation device generates the carbon nanotube by using the acetylene as a raw material.
